(19) United States
(12) Patent Application Publication
(10) Pub. No.: US 2026/0325277 A1
(43) Pub. Date: Oct. 22, 2026

(54) ABSORBABLE BORDER FOR HERNIA MESH

(71) Applicant: Hexagon Health, Inc., Beverly Hills, CA (US)

(72) Inventor: Shirin Towfigh, Beverly Hills, CA (US)

(21) Appl. No.: 19/486,338

(22) PCT Filed: May 24, 2024
(86) PCT No.: PCT/US2024/031144

§ 371 Date: Nov. 20, 2025

Related U.S. Application Data
(60) Provisional application No. 63/504,437, filed on May 25, 2023.

(51) Int. Cl. A61F 2/00

(52) U.S. Cl. CPC A61F 2/0063; A61F 2210/0004; A61F 2220/0008; A61F 2250/0031

(57) ABSTRACT

Disclosed herein are implantable meshes for inguinal hernia repair in a patient, comprising an absorbable border made to extend from the edges of a hernia mesh. In some embodiments, the implant comprises a mesh comprising a fabric layer configured to adhere to a tissue upon implantation of the mesh in the subject, the mesh is configured to cover at least a portion of a hernia, and a border composed of a bioabsorbable material, the border coupled to a portion of an edge of the mesh and extending at a non-zero angle therefrom, wherein the border is configured to be absorbed within a target amount of time after implantation.

### CROSS REFERENCE TO RELATED APPLICATIONS

This application is the U.S. National Stage entry of International Application No. PCT/US2024/031144, filed May 24, 2024. Which claims the benefit of and priority to U.S. Provisional Application No. 63/504,437, filed on May 25, 2023, the disclosure of each of which is hereby incorporated by reference in its entirety for all purposes. All publications, patents, and patent applications discussed herein are incorporated by reference herein to the same extent as if each individual publication, patent, or patent application was specifically and individually indicated to be incorporated by reference.

### BACKGROUND OF THE INVENTION

Hernia occurs when there is a weakness or hole in the muscular wall. Such hole allows organs and tissues to push through, or herniate, producing a bulge. One example is an inguinal hernia, which is a protrusion of the abdominal-cavity contents through the inguinal canal or other defects within the groin region. Inguinal hernia is common in men. Inguinal hernia repair with mesh is one of the most frequently performed operations.

### SUMMARY OF THE INVENTION

Systems, methods and devices described herein relate to an implant for hernia repair in a subject. In some embodiments, the implant comprises, a mesh comprising a fabric layer configured to adhere to a tissue upon implantation of the mesh in the subject, the mesh configured to cover at least a portion of a hernia, and a border composed of a bioabsorbable material, the border coupled to a portion of an edge of the mesh and extending at a non-zero angle therefrom, wherein the border is configured to be absorbed within a target amount of time after implantation. In some embodiments, the target amount of time is greater than an adhesion time for the mesh to at least partially adhere to the tissue surrounding the hernia. In some embodiments, the target amount of time is at least about 1 week, at least about 2 weeks, at least about 1 month, at least about 2 months, at least about 3 months, at least about 4 months, at least about 6 months, at least about 1 year, or at least about 2 years. In some embodiments, the border extends from an inferior edge of the mesh, a lateral edge of the mesh, a medial edge of the mesh, a superior edge of the mesh, or a combination of two or more thereof. In some embodiments, the border extends from the edge by at least about 1 mm, at least about 2 mm, at least about 5 mm, at least about 1 cm, at least about 2 cm, at least about 3 cm, at least about 5 cm, or at least about 10 cm. In some embodiments, the border extends outward from a first edge location of the edge of the mesh to form a first border width, wherein the border extends outward from a second edge location of the edge of the mesh spaced apart from the first location to form a second border width, and wherein a length of the border extends between the first border width and the second border width. In some embodiments, the border extends from the edge of the mesh at a non-zero angle relative to the mesh at a first edge location to form a first border width generally along a lateral edge of the border, and wherein the border extends from the edge of the mesh at a non-zero angle relative to the mesh at a second edge location to form a second border width generally along a medial edge of the border, and wherein a length of the border extends between the first border width and the second border width. In some embodiments, the first border width is longer than the second border width. In some embodiments, the first border width is the same length as the second border width. In some embodiments, the length forms a smooth gradation in extension from the mesh edge between the first border width and the second border width. In some embodiments, the border comprises alginate, carbonates (e.g., trimethylene carbonate, tetramethylene carbonate, and the like), chitosan, collagen, dioxanones (e.g., 1,4-dioxanone and p-dioxanone), fibrin, gelatin, hyaluronic acid, hydrophilic vinyl polymers, 2-2-methacryloyloxyethyl phosphorylcholine, hydroxamates, hydroxyalkanoate, polyhydroxybutyrate, polyhydroxyvalerate, poly(3-hydroxybutyrate-co-3-hydroxyvalerate, polyhydroxyoctanoate, and polyhydroxyhexanoate, icodextrin, polyester anyhydrides, polycarbonates, poly(3-hydroxybutyrate-co-3-hydroxyvalerate) (PHBV), poly(ethylene adipate) (PEA), poly(ethylene oxide-co-caprolactone) (PEOCL), poly(ethylene oxide-co-lactide) (PELA), poly(ethylene oxide-co-propylene oxide) (PEOPO), poly(ethylene oxide terephthalate) (PEOT), poly(ethylene succinate) (PES), poly(ethylene terephthalate) (PET), poly(ester-amide) (PEA), poly(ester-anhydride) (PEAN), poly(ester carbonate urethane) (PECU), poly(ester-ether-ester) (PEE), poly(glycolic acid-co-caprolactone) (PGACL), poly(glycolic acid-co-trimethylene carbonate) (PGATMC), poly(glycolic acid-co-caprolactone) (PGACL), poly(glycolic acid-co-trimethylene carbonate) (PGATMC), poly(glycerol sebacate) (PGS), poly(hydroxybutyric acid), poly(hydroxyvaleric acid), poly(L-lactide-co-D,L-lactide)/polylactic acid, poly(lactic acid-co-caprolactone) (PLACL), poly(lactic-co-glycolic acid) (PLGA), poly(propylene carbonate) (PPC), poly(propylene fumarate) (PPF), poly(propylene glycol) (PPG), poly(trimethylene carbonate) (PTMC), poly(vinyl pyrrolidone) (PVP), polyethylene glycol (PEG), polylactic acid (PLA), polyanhydrides, polyoxaesters, polytetrafluoroethylene (PTFE), polypseudo amino acids, polyortho esters, polyorthoesters, lactones, orthoesters, hydroxybutyrates, vinyl furanones, and copolymer, block copolymers, homopolymers, blends, and combinations of any two or more thereof. In some embodiments, the border comprises only absorbable materials, such that the border is fully absorbable. In some embodiments, the border is coupled to the mesh via glue, sewing, grips, sutures, staples, or a combination of two or more thereof. In some embodiments, the mesh is configured as an inguinal mesh, an open mesh, a lap mesh, a robotic mesh, an onlay mesh, a sublay mesh, an underlay mesh, or a combination of two or more thereof. In some embodiments, the mesh is configured to implanted at least partially within an abdomen of the subject, a flank of the subject, or other areas where hernias occur and need treatment with a hernia mesh. In some embodiments, the mesh further comprises a single fin having a fin edge extending at least partially inferiorly from the fabric layer. In some embodiments, the border comprises a medial edge extending from the mesh at the first edge location, lateral edge extending from the mesh at the second edge location, and a free edge therebetween, wherein the free edge is longer than the edge of the border coupled to the mesh from the first edge location to the second edge location. In some embodiments, the border comprises a medial edge extending from the mesh at the first edge location, lateral edge extending from the mesh at the second edge location, and a free edge therebetween, wherein the free edge is shorter than the edge of the border coupled to the mesh from the first edge location to the second edge location. In some embodiments, the border comprises a medial edge extending from the mesh at the first edge location, lateral edge extending from the mesh at the second edge location, and a free edge therebetween, wherein the free edge is about equal than the edge of the border coupled to the mesh from the first edge location to the second edge location. In some embodiments, at least one of the medial edge, the lateral edge, and the free edge of the border are a straight edge when flattened. In some embodiments, at least one of the medial edge, the lateral edge, and the free edge of the border form a curve when flattened. In some embodiments, at least one of the medial edge, the lateral edge, and the free edge of the border form an arc when flattened. In some embodiments, the border extends from the mesh only on the lateral half of the mesh. In some embodiments, the border extends from the mesh only on the medial half of the mesh. In some embodiments, the border extends from the mesh at least on the lateral half of the mesh. In some embodiments, the border extends from the mesh at least on the medial half of the mesh. In some embodiments, the first border width and the second border width are about equal. In some embodiments, the first border width and the second border width are each about 1 mm, at least about 2 mm, at least about 5 mm, at least about 1 cm, at least about 2 cm, at least about 3 cm, at least about 5 cm, or at least about 10 cm. In some embodiments, the first border width and the second border width are different. In some embodiments, a free edge border from the first location to the second location has a free edge border width that varies in width. In some embodiments, the free edge border width varies in width along its length about 1 mm, about 5 mm, about 1 cm, about 2 cm, about 3 cm, about 5 cm, about 10 cm, at least about 1 mm, at least about 2 mm, at least about 5 mm, at least about 1 cm, at least about 2 cm, at least about 3 cm, at least about 5 cm, or at least about 10 cm, from 1 cm to 10 cm, from 1 cm to 5 cm, from 2 cm to 5 cm, or from 1 cm to 3 cm. In some embodiments, the free edge border width along its length from the first location to the second location is about 1 mm, about 5 mm, about 1 cm, about 2 cm, about 3 cm, about 5 cm, about 10 cm, 1 mm to 10 cm, about 1 cm to about 10 cm, about 2 cm to about 10 cm, about 1 cm to about 5 cm, about 2 cm to about 5 cm, at least about 1 mm, at least about 2 mm, at least about 5 mm, at least about 1 cm, at least about 2 cm, at least about 3 cm, at least about 5 cm, at least about 10 cm, from 1 cm to 10 cm, from 1 cm to 5 cm, from 2 cm to 5 cm, from 1 cm to 3 cm, at least 1 mm, at least 2 mm, at least 5 mm, at least 1 cm, at least 2 cm, at least 3 cm, at least 5 cm, or at least 10 cm.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates an exemplary sublay left inguinal hernia mesh having an absorbable border that extends the inferior edge of the mesh, according to embodiments described herein.

FIG. 1B illustrates an example of the sublay left inguinal hernia mesh having an absorbable border that extends partially along and outwards from the inferior edge of the mesh, according to embodiments described herein.

FIG. 2A illustrates an exemplary left inguinal mesh, sublay position, having an absorbable border extending outwards from the inferior border of the original mesh, according to embodiments described herein.

FIG. 2B illustrates an example of the left inguinal mesh having an absorbable border that extends partially along and outwards or at a non-zero angle from the inferior edge of the mesh, according to embodiments described herein.

FIG. 3A illustrates a left onlay mesh with a fin for femoral hernia and inguinal repair, having an absorbable border extending along and outwards or at a non-zero angle from the inferior edge of the mesh, according to embodiments described herein.

FIG. 3B illustrates an example of the left onlay mesh with a fin for femoral hernia and inguinal repair having an absorbable border that extends partially along and outwards (or at a non-zero angle) from the inferior edge of the mesh, according to embodiments described herein.

FIG. 4A illustrates an example of a mesh for inguinal hernia repair having an absorbable border extending along and outwards (or at a non-zero angle) from the inferior edge, according to embodiments described herein.

FIG. 4B illustrates an example of the mesh for inguinal hernia repair having an absorbable border that extends partially along and outwards (or at a non-zero angle) from the inferior edge of the mesh, according to embodiments described herein.

### DETAILED DESCRIPTION OF THE INVENTION

As used herein, all numerical values or numerical ranges include whole integers within or encompassing such ranges and fractions of the values or the integers within or encompassing ranges unless the context clearly indicates otherwise. Thus, e.g., reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000-fold includes 1, 2, 3, 4, 5, 6, 7, 8, 9, 10, 11, 12, 13, 14, 15, 16, 17, 18, 19, 20-fold, etc., as well as 1.1, 1.2, 1.3, 1.4, 1.5-fold, etc., 2.1, 2.2, 2.3, 2.4, 2.5-fold, etc., and so forth.

As used herein the terms “3D” and “three-dimensional” refer to the shape a mesh and/or a border of a mesh conforms to. The mesh and border described herein are planar, flexible materials (e.g., having a fabric-like characteristics). For example, the mesh and border of the mesh may be placed over a sphere where the mesh will take the shape and curvature of a sphere while remaining a planar material. Similarly, if only the mesh portion is placed over a sphere, then the mesh portion would take on the shape of a sphere, while the border would extend in a different direction from the sphere.

There are various types of hernias known, some of which are location specific, and/or some of which are gender specific. For example, inguinal hernias are defects within the muscle and fascia planes of the groin region, wherein men are considered to be more prone to inguinal hernias due to the narrower and deeper male pelvis.

At least one method for hernia repair includes implanting a mesh about the defect, so as to contain the resulting bulge. The gold standard for hernia repair involves use of a mesh to patch the hernia defect in a tension-free manner. In some embodiments, hernia repair is performed using open, laparoscopic, or robotic-assisted techniques. The implantation of mesh has shown to be superior to non-mesh techniques due to the significantly lower risk of hernia recurrence when such a tension-free repair is performed. There are a numerous variety of mesh brands, sizes, and surgical techniques for mesh implantation. Meshes can include purely non-absorbable material, purely absorbable material, or a hybrid of absorbable and non-absorbable material.

Complications may however occur with mesh placement. The mesh can migrate, erode, fold, buckle, break, tear, disintegrate, or cause an autoimmune or inflammatory reaction. This folding of the mesh is referred to as a “meshoma.” This can cause hernia recurrence or chronic pain in various forms. Folding or buckling of the mesh is especially a problem with sublay or underlay positioning of the mesh deep to the inguinal floor muscle. This is commonly performed with the laparoscopic or robotic-assisted approaches. Such buckling or folding has been referred to as “taco-ing” or “clam shelling” of the mesh. Techniques related to the placement of the mesh can help reduce risks of movement, buckling, or folding of the mesh. One technique is to maximize the dissection of tissue in the retromuscular plane prior to mesh placement. Another is to allow for wider overlap of the mesh over normal healthy tissues.

Provided herein are meshes having a reduced footprint when implanted for hernia repair. Specifically, the meshes described herein include a border extending thereof that is implanted with the mesh and at least partially secures the mesh to the desired tissue, wherein at least a portion of the border is absorbable to the surrounding tissue, such that said absorbable portion is thereby removed from the mesh. Provided herein, therefore are devices comprising mesh coupled to biodegradable borders that address the issue of mesh folding or buckling in patients who undergo mesh implantation for inguinal hernia repair.

Mesh-related complications in the inguinal region may be a result of mesh folding, migration, and/or abnormal adherence or erosion into nearby structures. This can result in a hernia recurrence and/or may affect the nerves, vessels, vas deferens (in males), and/or bladder by inciting an abnormal interaction between the folded mesh and these nearby structures.

The meshes and methods disclosed herein are intended to help reduce mesh folding or migration from its original position by adding an absorbable or mostly absorbable component (e.g., a border as described herein) that will protect the mesh from folding during the initial stages of its integration with normal tissues. As used herein, bioabsorbable and absorbable are used interchangeably, and are intended to mean that such portion of the device is capable of being absorbed by the subject's body and/or degraded into smaller portions that are then removed from the body, and/or chemically and physically altered or broken down and cleared from the physiological environment by any means without outside intervention to do so. The absorbable component is intended to remain active during the healing stages after the operation, and while the mesh is integrating into the normal tissues. Once the mesh is fully integrated and can no longer move, the absorbable component may start disintegrating. The absorbable or mostly absorbable component may be synthetic, biologic, or a combination of both.

The meshes and methods disclosed herein may be used to reduce one or more selected from: chronic pain, functional impairment, and sexual dysfunction due to mesh folding. At the same time, the absorbable component of the inguinal hernia mesh may be used to reduce hernia recurrence and/or its severity. The devices disclosed herein may be applicable to various mesh placement techniques and surgical techniques. As non-limiting examples, these techniques include onlay mesh placement, sublay mesh placement, mesh-plug placement, retroperitoneal mesh placement, open mesh techniques, laparoscopic techniques, robotic techniques, or a combination thereof.

As used herein the terms “absorbed” and “absorption rate” when used to describe the border of the mesh refers to when the material of the border which has dissolved or degraded a target percentage within a target time period when tested ex vivo in solution. In some embodiments, the target percentage is 99%. In some embodiments, the target percentage is 95%. In some embodiments, the target percentage is 90%. In some embodiments, the target percentage is 85%. In some embodiments, the target percentage is 80%. In some embodiments, the target percentage is 75%. In some embodiments, the target percentage is 70%. In some embodiments, the target percentage is 65%. In some embodiments, the target percentage is 60%. In some embodiments, the target percentage is 55%. In some embodiments, the target percentage is 50%. In some embodiments, the target percentage is 45%. In some embodiments, the target percentage is 40%. In some embodiments, the target percentage is 35%. In some embodiments, the target percentage is 30%. In some embodiments, the target percentage is 25%. In some embodiments, the target percentage is 20%. In some embodiments, the target percentage is 15%. In some embodiments, the target percentage is 10%. In some embodiments, the target percentage is 5%.

In some embodiments, the absorption rate of the border is controlled by selecting a material of a desired thickness. For example, a thicker border may absorb at a slower rate than a thinner border. In some embodiments, the border thickness is between 0.1-0.5, 0.5-1.0, 1.0-1.5, 1.5-2.0, 2.0-2.5, 2.5-3.0, 3.0-3.5, 3.5-4.0, 4.0-4.5, 4.5-5.0, 5.0-5.5 millimeters. In some embodiments, the absorption rate of the border is controlled by altering the composition of polymers making up the border. In some embodiments, the absorption rate is controlled by the density of the border. For example, a porous border may be used for a faster absorption rate while a solid border may be used for a slower absorption rate.

In some embodiments, the absorption rate of the border is selected to substantially match the tissue integration rate into the mesh around the hernia so as to maintain adherence between the mesh and the tissue. In some embodiments, the border is selected to substantially match the tissue integration rate into the entire mesh so as to maintain adherence between the mesh and the tissue and prevent a portion or the entire mesh from curling within the patient.

Disclosed herein, in some aspects, is an implant for hernia repair in a subject, the implant comprising: a mesh comprising a fabric layer comprising a plurality of pores configured to enable tissue adhesion to the mesh upon implantation of the mesh in the subject, the mesh configured to cover at least a portion of the hernia; and a border extending from the mesh, the border configured to be at least partially absorbable into the subject after a target amount of time after implantation into the subject.

In some embodiments, the target amount of time corresponds to the mesh at least partially adhering to the subject about the portion of the hernia. In some embodiments, the target amount of time is at least about 1 week, at least about 2 weeks, at least about 1 month, at least about 2 months, at least about 3 months, at least about 4 months, at least about 6 months, at least about 1 year, or at least about 2 years.

In some embodiments, the border extends from any edge defining the mesh. In some embodiments, the border extends from a inferior edge of the mesh, a lateral edge of the mesh, a medial edge of the mesh, a superior edge of the mesh, or any combination thereof. In some embodiments, the border extends from at least a portion of the mesh by at least about 1 mm, at least about 2 mm, at least about 5 mm, at least about 1 cm, at least about 2 cm, at least about 3 cm, at least about 5 cm, or at least about 10 cm. In some embodiments, the border comprises one or more absorbable materials. In some embodiments, the border comprises only absorbable materials, such that the border is fully absorbable. In some embodiments, the border is coupled to the mesh via glue, sowing, grips, sutures, staples, or any combination thereof. In some embodiments, the mesh is configured as an inguinal mesh, an open mesh, a lap mesh, a robotic mesh, an onlay mesh, a sublay mesh, an underlay mesh, or any combination thereof. In some embodiments, the mesh is configured to implanted about an abdomen of the subject, a flank of the subject, or other areas thereabout. In some embodiments, the mesh further comprises a single fin having a fin edge extending at least partially inferiorly from the fabric layer.

The present invention relates to implants used for repairing a hernia. In some embodiments, repairing a hernia comprises implanting a mesh to overlay the hernia defect and optionally surrounding area. In some embodiments, any type of hernia mesh can be used, such as inguinal, abdominal/ventral, or any combination thereof. In some cases, inguinal hernias include all hernias of the groin. In some cases, inguinal hernia includes but is not limited to indirect and directs inguinal hernias, femoral hernias, and obturator hernias. Typically, implantable mesh (generally sheets of mesh having two flat sides) adheres to the subject's tissue on both flat sides following and/or during an inflammatory response to the implantation of the device. The goal of hernia repair is for this adhesion to occur on both flat sides of a mesh, however, depending on mesh design and surgical technique, the mesh may migrate or may not lay flat and may fold or buckle during the initial adhesion process. This can cause functional deterioration, pain, and other effects which are difficult to treat and can cause life-long debility. Furthermore, in some embodiments, an optimal mesh repair (e.g., of an inguinal hernia) is to have adhesion of the mesh onto the abdominal and pelvic floor structures along its entire perimeter. If the adherence is not perfect, then the mesh may lift off from the intended area of adherence, resulting in folding, buckling, or balling up of the mesh. In some cases, by adding an additional absorbable component to the mesh, spanning the outer perimeter of the mesh, this additional component can help reduce the risk of mesh lifting off at its edges.

Accordingly, in one aspect, disclosed herein, are implantable meshes for hernia repair that include a border extending from an edge of the mesh, wherein the border is at least partially absorbable. In some embodiments, the border is configured to further secure the mesh to the subject, and configured to be absorbed in the tissue after a certain amount of time, wherein such amount of time may correspond to an amount of time for the mesh to sufficiently adhere to the tissue about the hernia defect (e.g., via tissue integration or tissue adhesion). In some embodiments, the border improves the adherence and long term efficacy of the original mesh piece by optimizing the initial healing process. In some cases, by having the absorbable border, the overall need for mesh in the long term is reduced (compared to traditional mesh implants) and only have it for the short term.

In some embodiments, the mesh, scaffold, or other implant, whether synthetic or non-synthetic, used for the intention of inguinal hernia repair are herein referred as a group termed “mesh.” In some cases, implants as disclosed herein include but are not limited to meshes, biologics, allografts, xenografts, scaffolds, bioactive synthetic meshes, absorbable meshes, non-absorbable meshes, hybrid products, and implants made of other biomaterials. In some cases, for the simplicity of language, “mesh” is equivalent to “implant” as disclosed herein.

In some cases, an optimal mesh repair of an inguinal hernia is to have selective adhesion of the mesh onto the abdominal and pelvic floor structures that support the hernia defect which includes but are not limited to nearby muscles, fascias, and ligaments, without adhesion to critical structures including but not limited to nerves (including a genital nerve) and optionally the round ligament in female and the spermatic cord and its content and nerves (including a genital nerve) in males. In some cases, by adding an adhesion barrier to areas on the mesh that are at risk for adherence or tissue ingrowth, selective adhesion and optimal hernia repair are achieved.

In some cases, implants as disclosed herein include one or more selected from: a synthetic implant, a partially synthetic implant, and a non-synthetic implant. In some cases, implants as disclosed herein include but are not limited to meshes, biologics, allografts, xenografts, scaffolds, bioactive synthetic meshes, absorbable meshes, non-absorbable meshes, hybrid products, and implants made of other biomaterials.

The devices and methods disclosed herein may include mesh for implantation during different surgical procedures, for non-limiting examples, open inguinal surgery, laparoscopic surgery, or robotic-assisted surgery. The devices and methods disclosed herein may include mesh for implantation at different location, for non-limiting example, left or right of the midline of the body, on top of muscle, and/or behind the muscle. The technique for mesh implantation may be open, laparoscopic, or robotic-assisted, and this does not vary greatly between males and females. The onlay, sublay, or hybrid techniques may not be significantly variable between males and females. The purpose of this application is not to change the operative technique for inguinal hernia repair, rather to only improve the mesh implant to address gender-based differences in pelvic anatomy and its content and thus reduce complications.

In some cases, mesh that has poor or slow adhesion to its surroundings is more likely to fail, thereby hernia repair using mesh that is poorly adherent has a higher hernia recurrence rate, higher seroma rate, and the mesh is more likely to migrate. Thus, the devices disclosed herein, with or without a border (as described herein) may have an initial inflammatory stage in which the device may adhere to typically but not limited to muscle, ligament, or fascia, as possible, and may have relatively rapid and relatively strong tissue ingrowth from as much normal healthy vascular tissue.

In some cases, during the initial inflammation stage of the mesh, it also adheres to and is involved in tissue ingrowth with structures that can result in postoperative pain, discomfort, functional deterioration, and sexual impairment. In some cases, these structures include but are not limited to the spermatic cord and its contents as well as nearby nerves. Non-limiting example of these structures includes the genital branch of the genitofemoral nerve and the nerves along the vas deferens, which runs within the spermatic cord. In some cases, adherence of the mesh to undesired tissues or regions occurs with both flat and three-dimensional mesh designs. In some cases, such undesired adherence causes chronic groin pain, pelvic pain, and/or pain with sexual intercourse and orgasm. Males may also suffer from testicular pain and infertility.

In some embodiments, the border (as described herein) helps alleviate said risk of the initial inflammatory stage by providing additional security of the mesh to the surroundings. In some embodiments, the border is adapted to prevent separation and/or curling of a portion of the mesh away from the wall of a user. For example, the border may be adapted to provide additional adhesion over the bottom of the mesh. In some embodiments, the border is adapted to at least partially cover the peritoneal nerves. In some embodiments, the border is adapted to at least partially cover the spermatic chord. Covering nerves and/or the spermatic chord at least partially with the border may prevent pulling of the mesh on the nerve by the patient causing pain while maintaining adhesion between the mesh and the border. Maintaining adherence of the mesh to the tissue initially while the mesh adheres to the tissue about or around the hernia is important to ensure repair of the hernia. The curling of an inguinal hernia mesh may occur as a result from a combination of factors related to the mesh material, surgical technique, and patient-specific issues. Mesh used for hernia repairs must be lightweight and flexible for placement, but this also may cause the mesh to be prone to curling under tension and/or after being placed in the body. Further, insufficient fixation of the mesh to the tissue of the patient surrounding the hernia can cause the mesh to curl away from the tissue wall. Moreover, postoperative body movements, the healing process, and environmental factors like temperature changes can influence the mesh's stability and shape. Postoperative movements such as, coughing or straining can place stress on the mesh, potentially causing it to shift and curl away from the tissue wall. Additionally, the body's healing response such as an inflammatory response to the material and/or the formation of scar tissue during tissue ingrowth, can influence the position and shape of the mesh.

When an inguinal hernia mesh curls, it increases the risk of decreased effectiveness of the hernia repair. For example, a curled mesh may fail to adequately cover the defect in the abdominal wall, leading to incomplete or unstable repair, which significantly increases the risk of the hernia recurring. Additionally, if the mesh is placed in the wrong position or shifts to an undesirable location, it may irritate surrounding nerves, causing chronic pain, and potentially damage nearby tissues and organs. This irritation and damage not only result in discomfort but can also complicate post-surgical recovery and diminish the overall success of the hernia repair.

Moreover, a curled hernia mesh may increase the risk of infection and other complications. For example, the undesirable positioning may create spaces where fluids accumulate, fostering an environment for infections, which are serious complications that might necessitate removal of the mesh. Furthermore, adhesions may form if the mesh adheres to other adjacent tissues and/or organs, leading to chronic pain and potentially causing bowel obstruction. Furthermore, curling would likely result in poor integration of the mesh into the tissue and impede proper healing, diminishing the overall effectiveness of the repair. These issues highlight the critical need for meticulous surgical technique and the use of suitable mesh materials to ensure the mesh lies flat and remains securely in place during the tissue ingrowth time period.

In some embodiments, the mesh comprises of a fabric layer (as described herein, and as described in PCT application PCT/US2016/024090, which is incorporated herein by reference in its entirety).

In some embodiments, the implantable mesh for inguinal hernia repair disclosed herein is synthetic. In further embodiments, the fabric layer, the anti-adhesive barrier, or both the fabric layer and the anti-adhesive barrier are synthetic. In some embodiments, the synthetic mesh is made of one or more synthetic materials selected from but not limited to: polypropylene, polyester, expanded polytetrafluoroethylene (ePTFE), and copolymer, block copolymers, homopolymers, blends, and combinations of any two or more thereof and polymer suitable for inguinal floor reconstruction. In some embodiments the fabric layer comprises an interlaced structure defining a plurality of interstitial openings (alternatively called interstices herein).

In some embodiments, the border is composed of one or more bioabsorbable materials. In some embodiments, the synthetic mesh is made of one or more bioabsorbable materials selected from one or more selected from but not limited to: alginate, carbonates (e.g., trimethylene carbonate, tetramethylene carbonate, and the like), chitosan, collagen, dioxanones (e.g., 1,4-dioxanone and p-dioxanone), fibrin, gelatin, hyaluronic acid, hydrophilic vinyl polymers, 2-2-methacryloyloxyethyl phosphorylcholine, hydroxamates, hydroxyalkanoate, polyhydroxybutyrate, polyhydroxyvalerate, poly(3-hydroxybutyrate-co-3-hydroxyvalerate, polyhydroxyoctanoate, and polyhydroxyhexanoate, icodextrin, polyester anyhydrides, polycarbonates, poly(3-hydroxybutyrate-co-3-hydroxyvalerate) (PHBV), poly(ethylene adipate) (PEA), poly(ethylene oxide-co-caprolactone) (PEOCL), poly(ethylene oxide-co-lactide) (PELA), poly(ethylene oxide-co-propylene oxide) (PEOPO), poly(ethylene oxide terephthalate) (PEOT), poly(ethylene succinate) (PES), poly(ethylene terephthalate) (PET), poly(ester-amide) (PEA), poly(ester-anhydride) (PEAN), poly(ester carbonate urethane) (PECU), poly(ester-ether-ester) (PEE), poly(glycolic acid-co-caprolactone) (PGACL), poly(glycolic acid-co-trimethylene carbonate) (PGATMC), poly(glycolic acid-co-caprolactone) (PGACL), poly(glycolic acid-co-trimethylene carbonate) (PGATMC), poly(glycerol sebacate) (PGS), poly(hydroxybutyric acid), poly(hydroxyvaleric acid), poly(L-lactide-co-D,L-lactide)/polylactic acid, poly(lactic acid-co-caprolactone) (PLACL), poly(lactic-co-glycolic acid) (PLGA), poly(propylene carbonate) (PPC), poly(propylene fumarate) (PPF), poly(propylene glycol) (PPG), poly(trimethylene carbonate) (PTMC), poly(vinyl pyrrolidone) (PVP), polyethylene glycol (PEG), polylactic acid (PLA), polyanhydrides, polyoxaesters, polytetrafluoroethylene (PTFE), polypseudo amino acids, polyortho esters, polyorthoesters, lactones, orthoesters, hydroxybutyrates, vinyl furanones, and copolymer, block copolymers, homopolymers, blends, and combinations of any two or more thereof.

In some embodiments, the implantable mesh for hernia repair (any type of hernia repair, including inguinal hernia repair) disclosed herein is a hybrid mesh. In some embodiments, a hybrid mesh includes at least one synthetic material and at least one biological material. In further embodiments, the fabric layer, the anti-adhesive barrier, or both the fabric layer and the anti-adhesive barrier are hybrid. In some embodiments, the fabric layer is synthetic, biological or hybrid. In some embodiments, the fabric layer is hybrid, and the anti-adhesive barrier is biological, or hybrid, or synthetic. In some embodiments, the biological material is absorbable over a pre-determined period of time by the human body. In some embodiments, the implantable mesh for inguinal hernia repair disclosed herein is a biological mesh. In some embodiments, the implantable mesh for inguinal hernia repair disclosed herein is a hybrid mesh combining at least a biological and a synthetic material in either the fabric layer, the barrier, or both the fabric layer and the barrier. In some embodiments, the implantable mesh for inguinal hernia repair disclosed herein is a hybrid mesh combining at least an absorbable material and a synthetic material in either the fabric layer, a barrier, the attachment means of the barrier, both the fabric layer and the barrier, both the barrier and the attachment means, or all three of the fabric layer, the barrier, and the attachment means.

In some embodiments, the mesh may include one or more selected from but not limited to: a flat mesh, a 3-dimensional mesh, a preperitoneal mesh, an onlay mesh, a mesh plug, a Prolene hernia system (PHS) mesh, and a combination of these mesh categories.

In some embodiments, the mesh is intended to be positioned as a sublay or an onlay mesh. In some embodiments, the mesh is intended to positioned such that, for example, spermatic contents or a tube-like structure, lays on top of (or anteriorly to) the mesh, underneath (posterior to) the mesh, and/or through the mesh contacting the anterior side, the posterior side, or both sides of the mesh. In further embodiments, the surface, anterior, posterior, ventral, or dorsal surface of mesh that contact the tube like structure or the spermatic cord includes the barrier that protects the tissue from contacting the synthetic part of the mesh, for non-limiting example, the fabric layer.

In some embodiments, the mesh is individually packaged sterilely or non-sterilely.

In some cases, mesh braiding of the fabric layer may be variable. The width or length of the pore (see for example 101, 201, and 301 in PCT/US2016/024090, which is incorporated herein by reference in its entirety) may be any number between 0.001 mm to 10 mm or more. In some cases, the mesh pore may be any two-dimensional shape. For nonlimiting examples, the pore may be square, rectangular, round, oval, triangle, hexagon, pentagon, diamond, fan, or a combination thereof. In some cases, the pore size may be variable across the mesh. Alternatively, the fabric layer may not have a weave or a braid at all so that the fabric layer may be a flat sheet without visible pores or without any pores.

It should be understood, that dimensions sizes and shapes are exemplary and that any suitable sizes and shapes may be employed for the mesh.

In some cases, the mesh as described herein may be any shape that has a curved medial edge or a flat medial edge. The tip or peak of the medial curve may be pointing outward. The mesh may be any shape that has a substantially straight lateral edge or a curved lateral edge. In some cases, the mesh may be any shape that has a straight or curved inferior edge. In some cases, the mesh may be any shape that has a straight or curved superior edge. In some cases, the mesh may be any shape that has at least one smooth edge. In some cases, the mesh edges do not have any abrupt shape changes or transitions. As nonlimiting examples, the mesh has a shape of a square, a rectangle, a diamond, an oval, a trapezoid, a parallelogram, a round, a diamond, a u-shape, a bell-shape, a fan shape, a hexagon, a pentagon, or the like.

In some embodiments, for any mesh disclosed or as used herein, includes at least one fabric layer. The meshes as disclosed herein may only comprise a single fabric layer. The single fabric layer of a 2-dimensional mesh may comprise a surface area on one side of the fabric layer. The one side may be an anterior side, or a posterior side. The surface area on the anterior side and the surface area on the posterior side may be identical. The single fabric layer of a 3-dimensional mesh may comprise a surface are on multiple sides of the fabric layer. The sides may be an anterior, posterior, medial, and/or lateral side.

The surface area of the fabric layer may be the maximal area restricted by the edges of the fabric layer. The single fabric layer may have variation in its weave pattern, pore size, weight, density, and/or other physical features. The fabric layer may be adhesive to surrounding tissues. The fabric layer may be selectively anti-adhesive to spermatic cords, contents and/or critical nerves in the area. The fabric layer may be flat, folded, curved, or other 3-dimensional shape. The flat fabric layer may be folded, curved or bent under force. The flat fabric layer may be resiliently curved or bent. The fabric layer may be resiliently reshaped to different degrees from a flat fabric layer to accommodate the anatomy of different patients. The fabric layer may have a pre-determined resistance to reshaping in certain directions in order to hold the herniated tissue back from bulging.

The fabric layer may include a grid, a braid, a weave, an interlaced structure, or the like that defines a plurality of interstitial pores (e.g., 101, 201, 301 in PCT/US2016/024090). The pores may have a maximal dimension on the surface area that. The maximal dimension being a width, a length, a diameter, a long axis, or a diagonal. The maximal dimension may be no less than 5 mm, 4 mm, 3 mm, 2 mm, or 1 mm. Alternatively, the fabric layer may be sheet without any visible weave or any visible pores to naked eyes. The fabric layer may allow tissue adhesion to the mesh with tissue that is directly contacting one side or both side of the fabric layer. Such tissue adhesion may be nonselective. The grid, braid, weave of the fabric layer may enable tailoring of a keyhole and a slit to accommodate critical structures of the male or female patients without disruption or damage to the rest of the fabric layer and its physical properties.

The fabric layer may include a horizontal centerline. In some cases, the horizontal centerline is at exactly 50% of mesh height, the mesh height being in the superior-to-inferior direction, or the longitudinal direction of the mesh or of the patient upon implantation. Alternatively, the horizontal centerline may be at about 50% of mesh height, the mesh height being in the superior-to-inferior direction, or the longitudinal direction. The fabric layer may include a vertical centerline. In some cases, the vertical centerline is at exactly 50% of mesh width, the mesh height being in the medial-to-lateral direction, or the transverse direction of the mesh or of the patient upon implantation. Alternatively, the vertical centerline may be at about 50% of mesh width, the mesh width being in the medial-to-lateral direction, or the transverse direction.

The fabric layer may include an inferomedial region, quarter, corner, or edge. The inferomedial quarter may be the quadric of the surface area touching the inferior and medial edges of the fabric layer. Similarly, the inferolateral region may be the quadric touching the inferior and lateral edges of the fabric layer. A quadric, as described herein, may start from the tip point at exactly 50% mesh height and 50% mesh width and may fan out in an about 90-degree angle toward two adjacent edges of the surface area of the fabric layer. The two edges of the quadric starting from the tip point of the quadric may end when each edge touches one of two adjacent edges of the fabric layer. In further cases, the quadric may be bounded by its two edges at the midpoints of two adjacent edges of the fabric layer. The inferomedial corner may be a portion of the quadric touching the inferior and medial edges of the fabric layer. The inferolateral corner may be a portion of the quadric touching the inferior and lateral edges of the fabric layer.

The mesh height may be the maximal distance between a superior edge and an inferior edge in the longitudinal direction. The mesh width may be the maximal distance between a medial edge and a lateral edge in the transverse direction.

In some cases, the meshes as disclosed herein may include a fin. In some cases, a fin as disclosed herein may be added to the meshes as disclosed herein either during manufacturing or by a surgeon. If needed, the new mesh combining a mesh as disclosed herein and a fin as disclosed herein may be tailored to properly fit into various patients.

The mesh may have any shape or size that is suitable for onlay or sublay implantation for inguinal hernia. The maximal horizontal width of the mesh in the lateral-to-medial direction may be about 0.1 inch to about 15 inches. The maximal vertical height of the mesh in the superior-to-inferior direction may be about 0.5 inch to about 15 inches. Nonlimiting example of mesh sizes include about 1 inch by about 4 inches, about 2 inches by about 4 inches, about 3 inch by about 6 inches, about 4 inch by about 6 inches, about 3 inch by about 5 inches, about 5 inch by about 7 inches, about 1 inch by about 3 inches, about 2.5 inch by about 4.5 inches, or about 3.5 inch by about 5.5 inches.

In some embodiments, the mesh includes one or more selected from non-limiting list: a plug, a Prolene Hernia System® mesh, a Kugel mesh hernia patch °, and a Progeria mesh system®. In some embodiments, the mesh does not include a keyhole-like feature. In some embodiments, the barrier covers the contact area between the tube-like structure and the mesh.

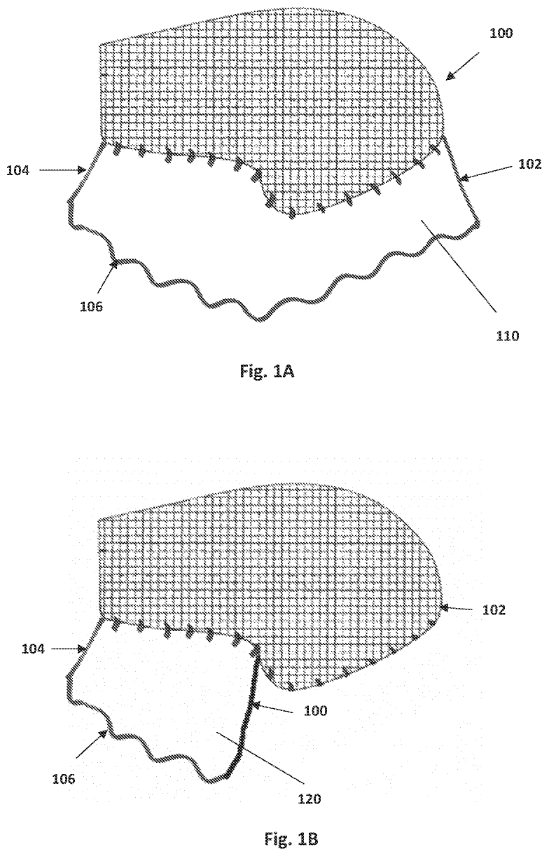

As described herein, for any mesh described herein, including as described in PCT/US2016/024090, a border may be provided that spans at least a portion of the outer perimeter of the mesh, including any fabric layer and/or fin (as described herein and in PCT/US2016/024090). In some embodiments, the border is configured to secure and/or provide additional securing support of the mesh to the subject (e.g., patient). For example, in some embodiments, the border is configured to be secured about a hernia defect and/or to the surrounding tissue thereof. In some embodiments, the border is configured to be attached to any portion of a mesh (as described herein). For example, in some embodiments, the border is configured to be attached to any mesh described herein at an inferior side, a superior side, a lateral side, a medial side, or a combination of two or more thereof. FIGS. 1 to 4 depict exemplary meshes (100, 200, 300, and 400, respectively) having a border (110, 210, 310, and 410, respectively) attached thereto at an inferior portion of the mesh.

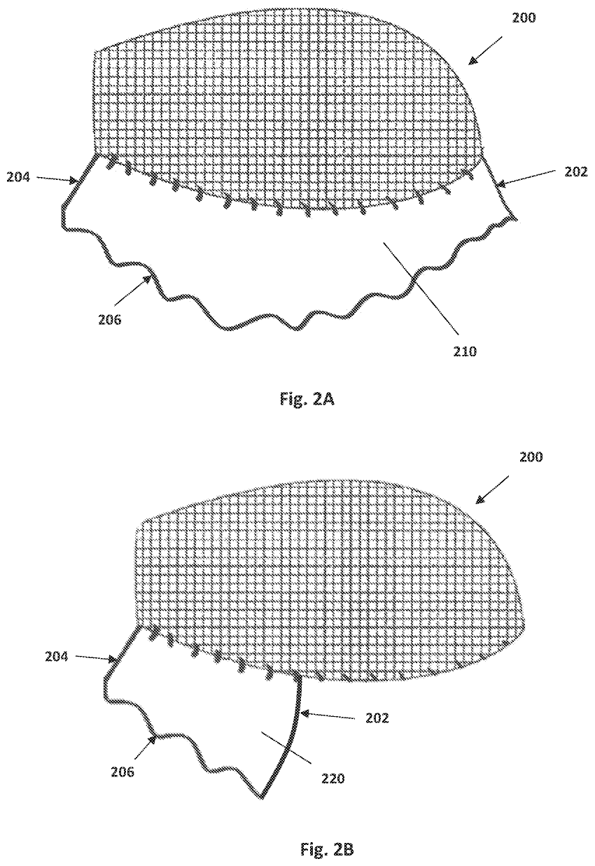

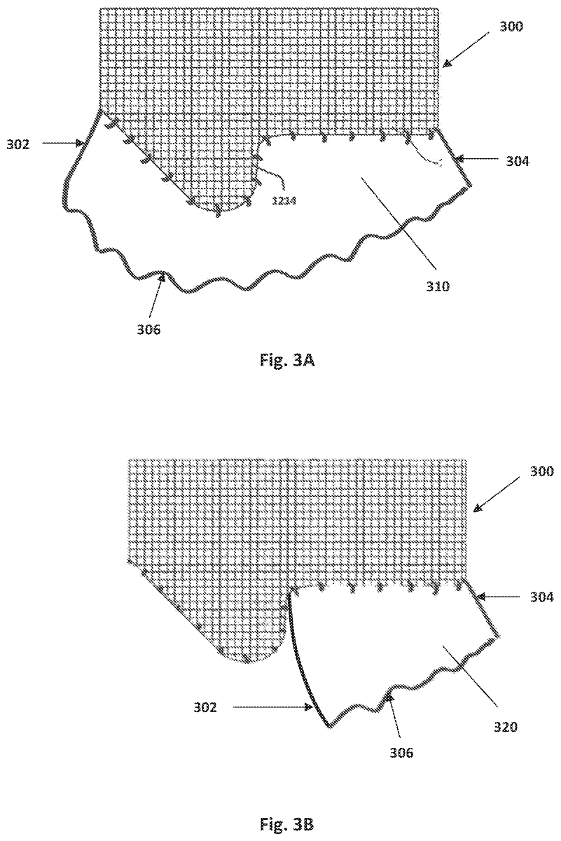

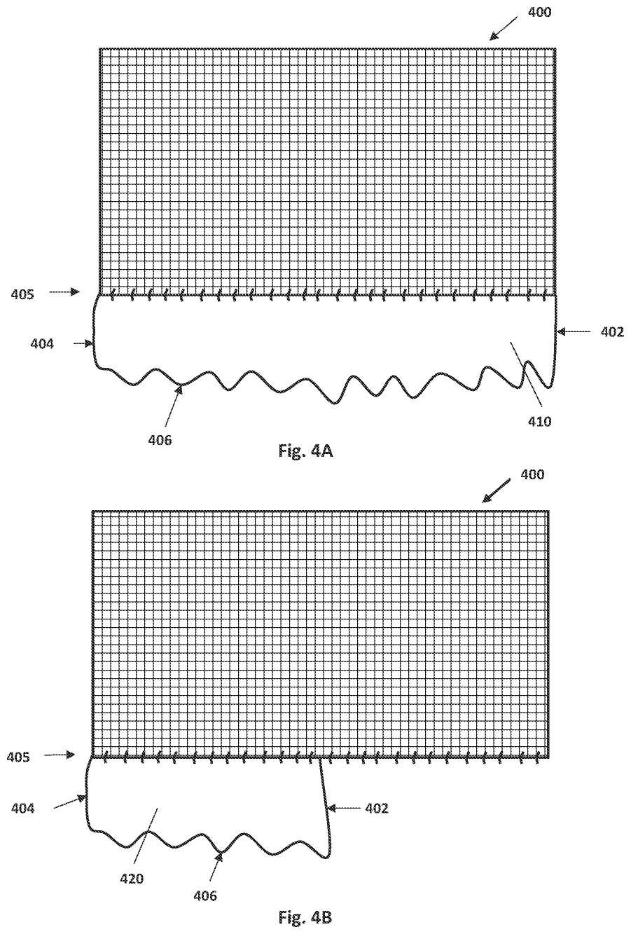

For example, FIG. 1A depicts a sublay left inguinal hernia mesh 100 having an absorbable border 110 that extends the inferior edge of the mesh. FIG. 2A depicts a left inguinal mesh 200 having an absorbable border 210 extending beyond the inferior border of the original mesh. FIG. 3A depicts a left onlay mesh 300 with a fin for femoral hernia and inguinal repair having an absorbable border 310 extending from the inferior edge of the mesh. FIG. 4A illustrates an example of an inguinal hernia repair mesh 400 for inguinal hernia repair having an absorbable border 410 extending along and outwards from the inferior edge 405. In some embodiments, any of the borders 120, 220, 230, and 420 extend around between 10-20%, 20-30%, 30-40%, 40-50%, 50-60%, 60-70%, 70-80%, 80-90%, 90-100% of the outer edge of the respective meshes 100, 200, 300, and 400.

FIG. 1B, FIG. 2B, FIG. 3B, and FIG. 4B illustrate exemplary embodiments where the border extends partially along a side of edge of the mesh. In some embodiments, the border extends from a portion of the edge of the mesh most likely to curl away from the tissue within the patient. For example, FIG. 1B illustrates an example of a border 120 extending outwards from the edge of the sublay left inguinal hernia mesh 100 and partially along the edge of the sublay left inguinal hernia mesh 100. In another example, FIG. 2B illustrates an example of a border 220 extending outwards from the edge of the left inguinal mesh 200 and partially along the edge of the left inguinal mesh 200. In another example illustrated in FIG. 3B, a border 320 extending outwards from the edge left onlay mesh 300 and partially along the edge of the left onlay mesh 300. In another example, as illustrated in FIG. 4B, a border 420 extending outwards from the inferior edge 405 of the inguinal hernia repair mesh 400 and partially along the edge of the inguinal hernia repair mesh 400.

In some embodiments, the border (110, 210, 310, and 410, respectively) comprises a medial edge (102, 202, 302, and 402, respectively) extending from the mesh at the first edge location, lateral edge (104, 204, 304, and 404, respectively) extending from the mesh at the second edge location, and a free edge (106, 206, 306 and 406, respectively) therebetween, wherein the free edge (106, 206, 306 and 406, respectively) is longer than the edge of the border coupled to the mesh from the first edge location to the second edge location. In some embodiments, the border comprises a medial edge (102, 202, 302, and 402, respectively) extending from the mesh at the first edge location, lateral edge (104, 204, 304, and 404, respectively) extending from the mesh at the second edge location, and a free edge (106, 206, 306 and 406, respectively) therebetween, wherein the free edge (106, 206, 306 and 406, respectively) is shorter than the edge of the border coupled to the mesh from the first edge location to the second edge location. In some embodiments, the border comprises a medial edge (102, 202, 302, and 402, respectively) extending from the mesh at the first edge location, lateral edge (104, 204, 304, and 404, respectively) extending from the mesh at the second edge location, and a free edge therebetween, wherein the free edge (106, 206, 306 and 406, respectively) is about equal than the edge of the border coupled to the mesh from the first edge location to the second edge location. In some embodiments, at least one of the medial edge (102, 202, 302, and 402, respectively), the lateral edge (104, 204, 304, and 404, respectively), and the free edge (106, 206, 306 and 406, respectively) of the border are a straight edge when flattened. In some embodiments, at least one of the medial edge (102, 202, 302, and 402, respectively), the lateral edge (104, 204, 304, and 404, respectively), and the free edge (106, 206, 306 and 406, respectively) of the border form a curve when flattened. In some embodiments, at least one of the medial edge (102, 202, 302, and 402, respectively), the lateral edge (104, 204, 304, and 404, respectively), and the free edge (106, 206, 306 and 406, respectively) of the border form an arc when flattened.

In some embodiments, the border is configured to be attached to the mesh by any attachment means known in the art, such as through stitching, sewing, weaving, gluing, spraying, other manufacturing methods for attachment, or any combination thereof. In some embodiments, the border extends from the mesh (e.g., at any edge of the mesh) at least about 0.1 cm to at least about 5 cm (relative to any portion of the mesh). In some embodiments, the border extends from the mesh at least about 0.5 cm, at least about 1 cm, at least about 1.5 cm, at least about 2 cm, at least about 2.5 cm, at least about 3 cm, at least about 4 cm, or at least about 5 cm.

In some embodiments, the border extends a variable distance from the mesh along the edge of the mesh. For example, the border may extend from a first location of the mesh to a first distance and extend along the edge of the mesh to a second location having a second distance from the mesh, wherein the first distance is greater that the second distance. For example, the distance of the border from the mesh at the first location may be a gradation in distance to the second location. In some embodiments, the distance in the width of the border between the first location and the second location is 0.1-0.5, 0.5-1, 1-1.5, 1.5-2, 2-2.5, 2.5-3, 3-3.5, 3.5-4, 4-4.5, or 4.5-5 mm.

In some embodiments, the border is configured to be secured to the subject (at and/or surrounding the hernia defect for example) via any means known in the art. For example, in some embodiments, the border is secured to the subject using one or more grips, a biocompatible adhesive, or comprises other material configured to be secured to the subject. In some embodiments, by being secured to the subject, and thereby securing and/or further securing the mesh about the hernia defect helps reduce the recurrence of a hernia as described herein (e.g., due to folding up of the mesh, thereby exposing the hernia defect).

In some embodiments, an absorbable border or partially absorbable border may be included about an inner edge of a keyhole for a mesh (e.g., for any keyhole described in a mesh in PCT/US2016/024090, including parastomal hernia mesh designs).

In some embodiments, the border is at least partially absorbable, such that at least a portion of the border is absorbed (e.g., by the tissue surrounding the hernia defect) and thereby removed from the mesh. In some embodiments, the border is configured to be at least partially absorbed after a minimum length of time after being implanted into the subject. For example, in some embodiments, the border is configured to be at least partially absorbed after at least about 1 day to about 1 year after implantation, at least about 1 week to about 6 months after implantation, at least about 2 weeks to about 4 months after implantation, or at least about 1 month to about 3 months after implantation. In some embodiments the border is configured to be at least partially absorbed after at least about 1 week, at least about 2 weeks, at least about 1 month, at least about 2 months, at least about 3 months, at least about 4 months, or at least about 6 months after implantation.

In some embodiments, the mesh is configured to be secured to the surrounding tissue (e.g., via tissue integration or tissue adhesion as described herein) by the time the border has been partially or fully absorbed, such that said border is no longer needed to keep the mesh in place and/or help prevent the mesh from folding up. In some embodiments, by configuring the border to be at least partially absorbed (e.g., by the surrounding tissue about the hernia defect), the amount of mesh required to be secured for the hernia repair can be reduced. For example, in some embodiments, the mesh can be sized to cover only the hernia defect, and/or a reduced portion thereabout (as compared to other traditional meshes), wherein the border will cover further portions of the surrounding tissue about the hernia. In such cases, when the border is absorbed, and thereby removed, the remaining mesh may be tailored to ensure coverage of the hernia defect. Such reduction in the total area covered by mesh may be beneficial for the subject, particularly in helping reduce the onset of post-implantation complications.

In some embodiments, the border is configured to be absorbed by comprising one or more absorbable materials (e.g., any absorbable material known in the art). In some embodiments, the border comprises only absorbable materials. In some embodiments, the border comprises a hybrid of one or more absorbable materials and one or more non-absorbable materials.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated. As used in this specification and the claims, unless otherwise stated, the term “about,” and “approximately” refers to variations of less than or equal to +/−1%, +/−2%, +/−3%, +/−4%, +/−5%, +/−6%, +/−7%, +/−8%, +/−9%, +/−10%, +/−11%, +/−12%, +/−14%, or +/−15%, depending on the embodiment. As a non-limiting example, about 100 meters represents a range of 95 meters to 105 meters, 90 meters to 110 meters, or 85 meters to 115 meters depending on the embodiments. The term “substantially” refers to less than or equal to +/−1%, +/−2%, +/−3%, +/−4%, +/−5%, +/−6%, +/−7%, +/−8%, +/−9%, +/−10%, +/−11%, +/−12%, +/−14%, or +/−15% variation. As a non-limiting example, substantially parallel represents a range of −1 to 1 degree difference, −5 to 5 degree difference, or −15 degrees to 15 degrees of difference from being parallel, depending on the embodiments.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

All publications, patents, and patent applications mentioned in this specification are herein incorporated by reference to the same extent as if each individual publication, patent, or patent application was specifically and individually indicated to be incorporated by reference.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An implant for hernia repair in a subject, the implant comprising:
a mesh having a first edge, the mesh comprising a fabric layer configured to adhere to a tissue upon implantation of the mesh in the subject and configured to cover at least a portion of a hernia; and
a border composed of a bioabsorbable material coupled to the mesh along the first edge of the mesh from a first edge location to a second edge location forming a border edge therebetween that extends the border from the mesh at a non-zero angle relative to mesh at its first edge, wherein the border is configured to be absorbed within a target amount of time after implantation.

2. The implant of claim 1, wherein the first edge is at least one of an inferior edge of the mesh, a superior edge of the mesh, a lateral edge of a mesh, and a medial edge of the mesh.

3. The implant of claim 1, wherein the target amount of time is at least about 1 week, at least about 2 weeks, at least about 1 month, at least about 2 months, at least about 3 months, at least about 4 months, at least about 6 months, at least about 1 year, or at least about 2 years.

4. The implant of claim 1, wherein the border has a medial edge extending from the first edge location away from the first edge of the mesh and a lateral edge extending from the second edge location away from the first edge of the mesh and a free edge extending from a second end of the medial edge to a second end of the lateral edge of the border.

5. The implant of claim 4, wherein the border extends from the first edge of the mesh to the free edge of the border by at least about 1 mm, at least about 2 mm, at least about 5 mm, at least about 1 cm. at least about 2 cm, at least about 3 cm, at least about 5 cm, or at least about 10 cm.

6-7. (canceled)

8. The implant of claim 4, wherein a first border width along the medial edge of the border is longer than a second border width along the lateral edge of the border.

9. The implant of claim 4, wherein a first border width along the medial edge of the border is the same length as a second border width along the lateral edge of the border.

10. The implant of claim 4, wherein a first border width along the medial edge of the border is shorter than a second border width along the lateral edge of the border.

11. The mesh claim 1, wherein the border comprises alginate, carbonates (e.g., trimethylene carbonate, tetramethylene carbonate, and the like), chitosan, collagen, dioxanones (e.g., 1,4-dioxanone and p-dioxanone), fibrin, gelatin, hyaluronic acid, hydrophilic vinyl polymers, 2-2-methacryloyloxy ethyl phosphorylcholine, hydroxamates, hydroxyalkanoate, polyhydroxybutyrate, polyhydroxy valerate, poly(3-hydroxybutyrate-co-3-hydroxy valerate, polyhydroxy octanoate, and polyhydroxyhexanoate, icodextrin, polyester anyhydrides, polycarbonates, poly(3-hydroxybutyrate-co-3-hydroxy valerate) (PHBV), poly(ethylene adipate) (PEA), poly(ethylene oxide-co-caprolactone) (PEOCL), poly(ethylene oxide-co-lactide) (PELA), poly(ethylene oxide-co-propylene oxide) (PEOPO), poly(ethylene oxide terephthalate) (PEOT), poly(ethylene succinate) (PES), polyethylene terephthalate) (PET), poly(ester-amide) (PEA), poly(ester-anhydride) (PEAN), poly(ester carbonate urethane) (PECU), poly(ester-ether-ester) (PEE), poly(gly colic acid-co-caprolactone) (PGACL), poly(gly colic acid-co-trimethylene carbonate) (PGATMC), poly(glycolic acid-co-caprolactone) (PGACL), poly(glycolic acid-co-trimethylene carbonate) (PGATMC), poly(glycerol sebacate) (PGS), poly(hydroxybutyric acid), poly(hydroxyvaleric acid), poly(L-lactide-co-D,L-lactide)/polylactic acid, poly(lactic acid-co-caprolactone) (PLACE), poly(lactic-co-glycolic acid) (PLGA), polypropylene carbonate) (PPC), polypropylene fumarate) (PPF), polypropylene glycol) (PPG), poly(trimethylene carbonate) (PTMC), poly(vinyl pyrrolidone) (PVP), polyethylene glycol (PEG), polylactic acid (PLA), polyanhydrides, poly oxaesters, polytetrafluoroethylene (PTFE), polypseudo amino acids, polyortho esters, polyorthoesters, lactones, orthoesters, hydroxybutyrates, vinyl furanones, and copolymer, block copolymers, homopolymers, blends, and combinations of any two or more thereof.

12. The mesh of claim 1, wherein the border comprises only absorbable materials, such that the border is fully absorbable.

13. The mesh of claim 1, wherein the border edge is coupled to the first edge of the mesh via glue, sewing, grips, sutures, staples, or a combination of two or more thereof.

14. The mesh of claim 1, wherein the first edge of the mesh is an inferior edge of the mesh such that upon implantation, the border edge is adapted to at least partially cover the peritoneal nerves or the spermatic chord to prevent pulling of the mesh on the nerve by the patient causing pain while maintaining adhesion between the mesh and tissue.

15. The mesh of claim 1, wherein the border is configured to reduce or prevent folding, buckling, or migration of the mesh during adhesion of the mesh following implantation.

16. The mesh of claim 1, wherein the mesh further comprises a single fin having a fin edge extending at least partially inferiorly from the fabric layer.

17. The mesh of claim 1, wherein the border comprises a medial edge extending from the mesh at the first edge location, lateral edge extending from the mesh at the second edge location, and a free edge therebetween, wherein the free edge is longer than the border edge.

18. The mesh of claim 1, wherein the border comprises a medial edge extending from the mesh at the first edge location, lateral edge extending from the mesh at the second edge location, and a free edge therebetween, wherein the free edge is shorter than the border edge.

19. The mesh of claim 1, wherein the border comprises a medial edge extending from the mesh at the first edge location, lateral edge extending from the mesh at the second edge location, and a free edge therebetween, wherein the free edge is about equal than the border edge.

20. The mesh of claim 17, wherein at least one of the medial edge, the lateral edge, and the free edge of the border are a straight edge when flattened.

21. The mesh of claim 17, wherein at least one of the medial edge, the lateral edge, and the free edge of the border form a curve when flattened.

22. (canceled)

23. The mesh of claim 1, wherein the border extends from the mesh only on a lateral half of the mesh.

24. The mesh of claim 1, wherein the border extends from the mesh only on a medial half of the mesh.

25. The mesh of claim 1, wherein the border extends from the mesh at least on a lateral half of the mesh.

26. The mesh of claim 1, wherein the border extends from the mesh at least on a medial half of the mesh.

27. The mesh of claim 1, wherein a plane of the border extends in a different direction than a plane of the mesh.

28. The mesh of claim 4, wherein a first border width from the first edge location to the free edge and a second border width from the second edge location to the free edge are each about 1 mm, at least about 2 mm, at least about 5 mm, at least about 1 cm, at least about 2 cm, at least about 3 cm, at least about 5 cm, or at least about 10 cm.

29. The mesh of claim 28, wherein the first border width and the second border width are different.

30. The mesh of claim 4, wherein a width of the border varies along its free edge length measured from the free edge to the closest location on the first edge to which the border is attached.

31. The mesh of claim 30, wherein the width of the border varies in width along its length about 1 mm, about 5 mm, about 1 cm, about 2 cm, about 3 cm, about 5 cm, about 10 cm, at least about 1 mm, at least about 2 mm, at least about 5 mm, at least about 1 cm, at least about 2 cm, at least about 3 cm, at least about 5 cm, or at least about 10 cm, from 1 cm to 10 cm, from 1 cm to 5 cm, from 2 cm to 5 cm, or from 1 cm to 3 cm.

32. The mesh of claim 30, wherein the width of the border is about 1 mm, about 5 mm, about 1 cm, about 2 cm, about 3 cm, about 5 cm, about 10 cm, 1 mm to 10 cm, about 1 cm to about 10 cm, about 2 cm to about 10 cm, about 1 cm to about 5 cm, about 2 cm to about 5 cm, at least about 1 mm, at least about 2 mm, at least about 5 mm, at least about 1 cm. at least about 2 cm, at least about 3 cm, at least about 5 cm, at least about 10 cm, from 1 cm to 10 cm, from 1 cm to 5 cm, from 2 cm to 5 cm, from 1 cm to 3 cm, at least 1 mm, at least 2 mm, at least 5 mm, at least 1 cm, at least 2 cm, at least 3 cm, at least 5 cm. or at least 10 cm.
